(19) United States
(12) Patent Application Publication
(10) Pub. No.: US 2022/0211991 A1
(43) Pub. Date: Jul. 7, 2022

(54) ARTERIOTOMY POSITIONING DEVICE AND METHOD OF USE THEREFOR

(71) Applicant: Access Closure, Inc., Santa Clara, CA (US)

(72) Inventors: Eric Christopher DAILEY, Santa Clara, CA (US); Sravanthi AVUTHU, Portola Valley, CA (US)

(21) Appl. No.: 17/449,005

(22) Filed: Sep. 27, 2021

Related U.S. Application Data
(63) Continuation of application No. 16/131,611, filed on Sep. 14, 2018, now abandoned.
(60) Provisional application No. 62/558,462, filed on Sep. 14, 2017.

(51) Int. Cl. A61M 39/02; A61B 17/34; A61M 25/10; A61B 17/00

(52) U.S. Cl. CPC A61M 39/0247; A61B 17/3421; A61M 25/10182; A61M 2025/09075; A61B 2017/3486; A61M 2039/0258; A61B 17/0057

(57) ABSTRACT

A method and device for positioning an expandable support in a blood vessel is disclosed herein. The device may include a handle, a catheter assembly, a core wire, and an expandable support. The expandable support may be used to position the device relative to the arteriotomy, as well as to provide temporary hemostasis. The expandable support may be attached to the catheter assembly at a first end, and to the core wire at a second end to optimize the width of the expandable support based on the function being performed by the expandable support at a given time. The catheter assembly may be able to move relative to the handle and the core wire, thereby moving the first end of the expandable support relative to the handle and the core wire.

### CROSS-REFERENCE TO RELATED APPLICATIONS

This application claims the benefit of U.S. Provisional Patent Application No. 62/558,462, filed Sep. 14, 2017, which is incorporated herein by reference in its entirety.

### FIELD

Arteriotomy positioning devices described herein may be useful when performing diagnostic or therapeutic procedures requiring vascular access. The devices may be used to position an expandable support relative to an arteriotomy in a blood vessel.

### BACKGROUND

Some diagnostic or therapeutic procedures require access to a patient's vasculature (e.g., imaging procedure, angioplasty, stent delivery, or otherwise). To access the patient's vasculature percutaneously, a hollow needle may be inserted through a patient's skin and overlying tissue into a blood vessel. A guide wire may be passed through the needle lumen into the blood vessel, whereupon the needle may be removed. An introducer sheath may then be advanced over the guide wire into the vessel in conjunction with or subsequent to one or more dilators. A catheter or other device may be advanced through the introducer sheath and over the guide wire into a position for performing a medical procedure.

After completion of the diagnostic or therapeutic procedure requiring access to the vasculature, the arteriotomy can be closed by various mechanical or biological solutions, such as by applying external pressure (e.g., manually and/or using sandbags), cinching, suturing, and/or delivering metal implants, plugs, or sealants. However, many of these closure procedures may be time consuming, expensive, and uncomfortable for the patient, requiring the patient to remain immobilized in the operating room, catheter lab, or holding area for long periods of time. Additionally, some of these prolonged closure procedures may increase the risk of hematoma from bleeding prior to hemostasis.

When closing the arteriotomy using a metal implant, plug, sealant, or other appropriate sealing member, the health care professional may use a vascular closure device to position and deploy the sealing member. The vascular closure device may include a balloon near the distal end of the device to aid in positioning the sealant relative to the arteriotomy. The balloon is not inflated when the device is provided. The distal end of the device is inserted into the puncture until the uninflated balloon is positioned in the vessel. The balloon is then inflated, and the user can verify inflation by many methods, one including viewing an inflation indicator on a proximal end of the device. The device can then be positioned by withdrawing the device proximally until the inflated balloon contacts the vessel wall around the arteriotomy, indicating that the sealant is in the correct position. Such an indication is often provided by tactile feedback. Once the balloon contacts the vessel wall, the health care professional may continue pulling back on the device while the balloon remains inflated to apply a proximal force to the vessel wall. In existing devices, applying a proximal force to the vessel wall does not substantially change the shape of the balloon.

### SUMMARY

Arteriotomy positioning devices described herein may be used to position a sealing member adjacent to an arteriotomy. The arteriotomy positioning device may have a handle, a catheter assembly, a core wire, and an expandable member. The core wire may be substantially fixed relative to the handle, at least while the device is in the locating and/or tension states (described below). The catheter assembly may be moveable relative to the handle and the core wire. The proximal end of the expandable support may be connected to the catheter assembly, and the distal end of the expandable support may be connected to the core wire. The distance between distal end of the expandable support and a distal end of the handle may be substantially fixed, at least while the device is in the locating and/or tension states, whereas the proximal end of the expandable support may be moveable relative to the handle. The proximal end of the expandable support may be biased proximally, but moveable distally relative to the handle in response to various forces applied to the device.

The arteriotomy positioning device may have several states, including a resting state, a locating state, and a tension state. In the resting state, the expandable support may have a width small enough to fit through the arteriotomy. In the locating state, the expandable support may have a width large enough that t does not fit through the arteriotomy, but small enough that it can move freely through small vessels, thereby reducing the chance that the expandable support will interact with bifurcations or calcifications in the blood vessel while the user is moving the expandable support into position adjacent the arteriotomy. Finally, in the tension state, the expandable support may be wider than it is when the device is in the resting and locating states in order to occlude the arteriotomy and create temporary hemostasis. Because the width of the expandable support may vary between the locating state and the tension state, the width of the expandable support may be optimized based on the function being performed by the expandable support at a given time. The expandable support may be narrow enough when locating the device relative to the arteriotomy in order to fit through small vessels, but wide enough when tension is being applied to occlude the arteriotomy and create temporary hemostasis.

Another advantage of this arteriotomy positioning device is that the device status indicator can provide several indications to the user. The device status indicator can indicate if the expandable support is in a low-profile, medium-profile, or high-profile configuration. The device status indicator can also indicate if tension is being applied to the catheter assembly, and if so, if the amount of tension being applied is appropriate. The device status indicator can also indicate when the device is in the correct position relative to the arteriotomy, since the device will show that tension is being applied once the expandable member is pulled against the vessel wall. For example, if the expandable support is a balloon, the device status indicator can indicate whether the balloon is inflated, whether the device is in the correct position relative to the arteriotomy, and/or whether tension is being applied to the catheter assembly (and if so, whether the amount of tension being applied is appropriate).

An exemplary device for positioning an expandable support may comprise a handle; a catheter assembly having a lumen, the catheter assembly extending from the handle; a core wire extending from the handle through the lumen of the catheter assembly, the core wire having a proximal end connected to the handle and a distal end extending from a distal end of the catheter assembly; and an expandable support having a proximal end connected to a distal end of the catheter assembly and a distal end connected to the distal end of the core wire; wherein the catheter assembly is slidable relative to both the handle and the core wire. The expandable member may be moveable between a low-profile configuration, a medium-profile configuration, and a high-profile configuration. The device may further comprise a device status indicator that indicates whether the expandable support is in the low-profile configuration, the medium-profile configuration, or the high-profile configuration; and whether tension is being applied to the catheter assembly. A distance between the distal end of the handle and the distal end of the catheter assembly may increase as the expandable member moves from the low-profile configuration to the medium-profile configuration, and the distance further increases as the expandable member moves from the medium-profile configuration to the high-profile configuration. A distance between the distal end of the handle and the distal end of the core wire may remain substantially constant as the expandable member moves between the low-profile configuration, the medium-profile configuration, and the high-profile configuration. The device may further comprise a spring positioned in the handle, wherein the spring applies a proximal force to the catheter assembly relative to the handle and the core wire. The handle may comprise a fluid chamber, and an inflation port that allows communication with the fluid chamber. The lumen of the catheter assembly may communicate with the fluid chamber. The catheter assembly may comprise a catheter and a plunger. The catheter assembly may comprise a resting stop that limits proximal movement of the catheter assembly relative to the handle and the core wire. The catheter assembly may comprise a tension stop that limits proximal movement of the handle and the core wire relative to the catheter assembly.

An exemplary method for positioning a device adjacent to an arteriotomy of a blood vessel may comprise inserting a distal end of a device into the blood vessel, the device comprising a core wire connected to a handle, a catheter assembly slidable relative to the handle and the core wire, and an expandable support having a length, a width, a proximal end connected to the catheter assembly, and a distal end connected to the core wire, wherein the expandable support is in a low-profile configuration; increasing the width of the expandable support and decreasing the length of the expandable support, thereby moving the expandable support from the low-profile configuration to a medium-profile configuration; withdrawing the device proximally until the expandable support contacts a wall of the blood vessel adjacent to the arteriotomy; and applying tension to the catheter assembly to further increase the width and decrease the length of the expandable support and bring the expandable support to a high-profile configuration. The relative positions of the handle and the core wire may remain substantially constant during the steps of withdrawing the device proximally, and applying tension to the catheter assembly. The step of bringing the expandable support to the medium-profile configuration may cause the catheter assembly to move distally relative to the core wire. The step of applying tension to the catheter assembly may cause the core wire to move proximally relative to the catheter assembly. The device may comprise a visual indicator having an indicator feature and a series of indicator markings, and wherein the step of bringing the expandable support to the medium-profile configuration may cause the indicator feature to move relative to the series of indicator markings. The step of applying tension to the catheter assembly may cause the indicator feature to move relative to the series of indicator markings. The expandable support may comprise a balloon, and the width may be a maximum diameter of the balloon. The step of increasing the width of the expandable support and decreasing the length of the expandable support may comprise inflating the expandable support by pushing an inflation fluid through the catheter assembly and into the expandable support. The method may further comprise performing a procedure returning the device to the low-profile configuration; and withdrawing the device from the blood vessel. The procedure may be a vascular closure procedure.

### BRIEF DESCRIPTION OF THE FIGURES

FIG. 1A is a perspective view of an arteriotomy positioning device in a resting state.

FIG. 1B is a cross-section view of the handle of the arteriotomy positioning device of FIG. 1A in a resting state, shown from the side.

FIG. 10 is a cross-section view of the expandable support of the arteriotomy positioning device of FIG. 1A in a resting state, shown from the side.

FIG. 2A is a perspective view of the arteriotomy positioning device in a locating state.

FIG. 2B is a cross-section view of the handle of the arteriotomy positioning device of FIG. 2A in a locating state, shown from the side.

FIG. 2C is a cross-section view of the expandable support of the arteriotomy positioning device of FIG. 2A in a locating state, shown from the side.

FIG. 3A is a perspective view of the arteriotomy positioning device in a tension state with too little force.

FIG. 3B is a cross-section view of the handle of the arteriotomy positioning device of FIG. 3A in a tension state with too little force, shown from the side.

FIG. 3C is a cross-section view of the expandable support of the arteriotomy positioning device of FIG. 3A in a tension state with too little force, shown from the side,

FIG. 4A is a perspective view of the arteriotomy positioning device in a tension state with an appropriate amount of force.

FIG. 4B is a cross-section view of the handle of the arteriotomy positioning device of FIG. 4A in a tension state with an appropriate amount of force, shown from the side.

FIG. 4C is a cross-section view of the expandable support of the arteriotomy positioning device of FIG. 4A in a tension state with an appropriate amount of force, shown from the side.

FIG. 5A is a perspective view of the arteriotomy positioning device in a tension state with too much force.

FIG. 5B is a cross-section view of the handle of the arteriotomy positioning device of FIG. 5A in a tension state with too much force, shown from the side.

FIG. 5C is a cross-section view of the expandable support of the arteriotomy positioning device of FIG. 5A in a tension state with too much force, shown from the side.

FIGS. 6-11 show a cross-section view of alternative embodiments of the handle of the arteriotomy positioning device in a resting state, shown from the side.

FIG. 12A is a perspective view of an alternative embodiment for an arteriotomy positioning device in a resting state.

FIG. 12B is a perspective view of the arteriotomy positioning device of FIG. 12A in a locating state.

FIG. 12C is a perspective view of the arteriotomy positioning device of FIG. 12A in an expanded state.

FIG. 13A is a cross-section view of a handle used with the expandable support of FIG. 12A in a resting state, shown from the side.

FIG. 13B is a cross-section view of the handle of FIG. 13A used with the expandable support of FIG. 12B in a locating state, shown from the side.

FIG. 13C is a cross-section view of the handle of FIG. 13A used with the expandable support of FIG. 12C in an expanded state, shown from the side.

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

### DETAILED DESCRIPTION

The detailed description set forth below, in connection with the appended drawings, is intended as a description of various configurations and is not intended to represent the only configurations in which the concepts described herein may be practiced. The detailed description includes specific details for the purpose of providing a thorough understanding of the various concepts. However, it will be apparent to those skilled in the art that these concepts may be practiced without these specific details.

Various aspects of an arteriotomy positioning device may be illustrated by describing components that are coupled, attached; connected, pneumatically associated, and/or joined together. As used herein, the terms “coupled”, “attached”, “connected”, “pneumatically associated”, “in communication with”, and/or “joined” are interchangeably used to indicate either a direct connection between two components or, where appropriate, an indirect connection to one another through intervening or intermediate components. In contrast, when a component is referred to as being “directly coupled”, “directly attached”, “directly connected” and/or “directly joined” to another component there are no intervening elements shown in said examples.

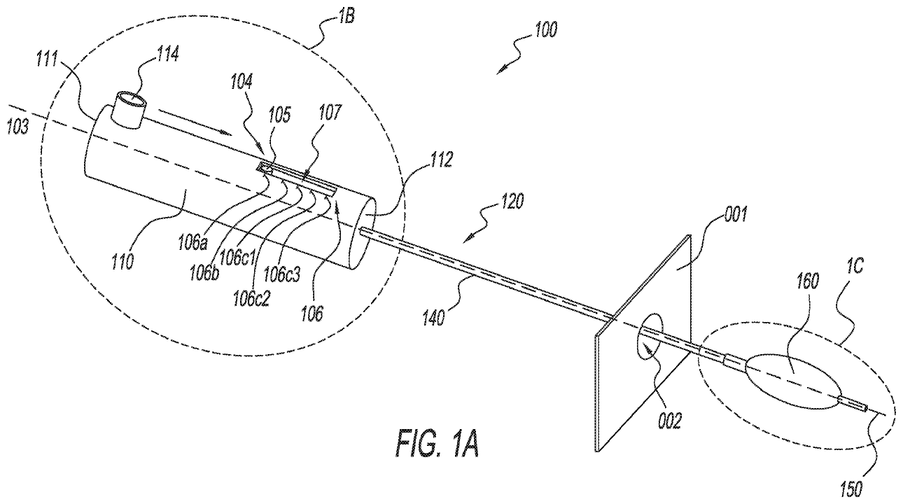

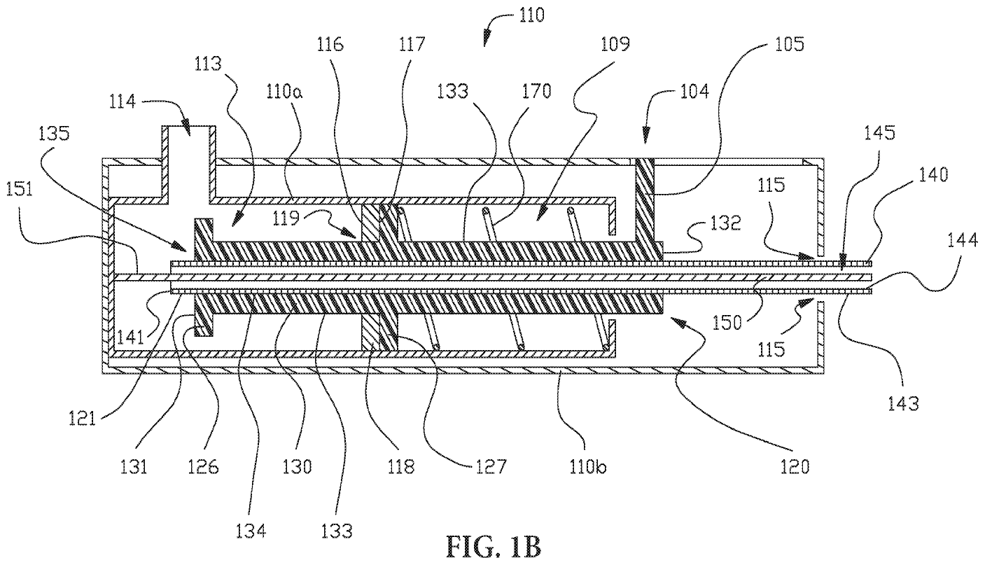

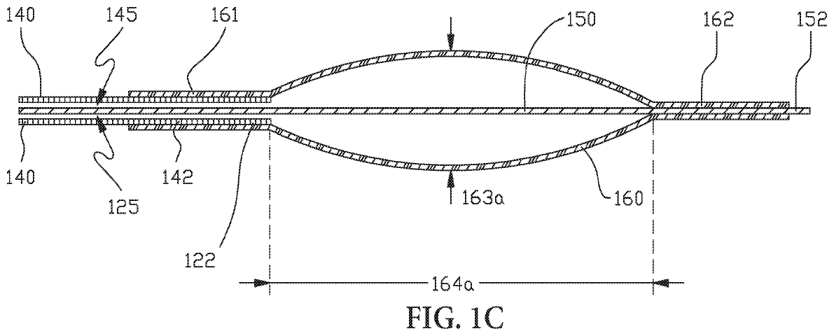

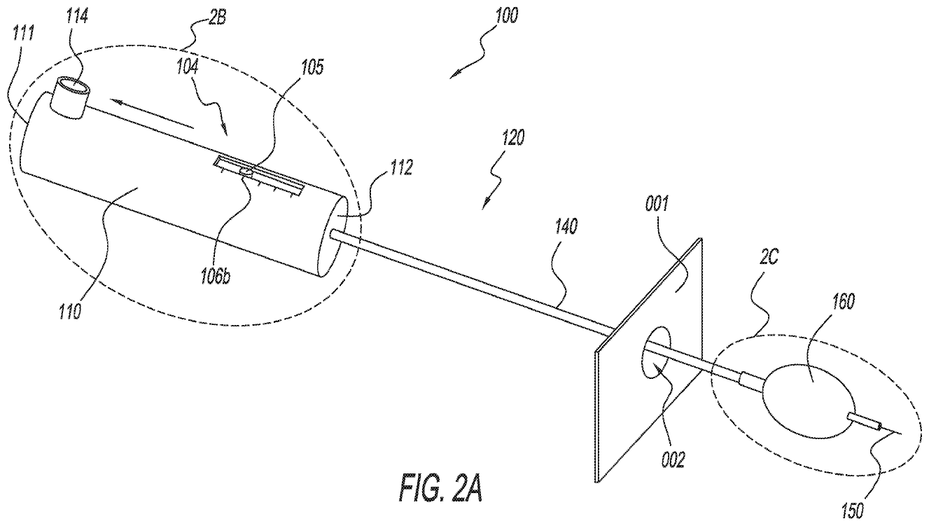

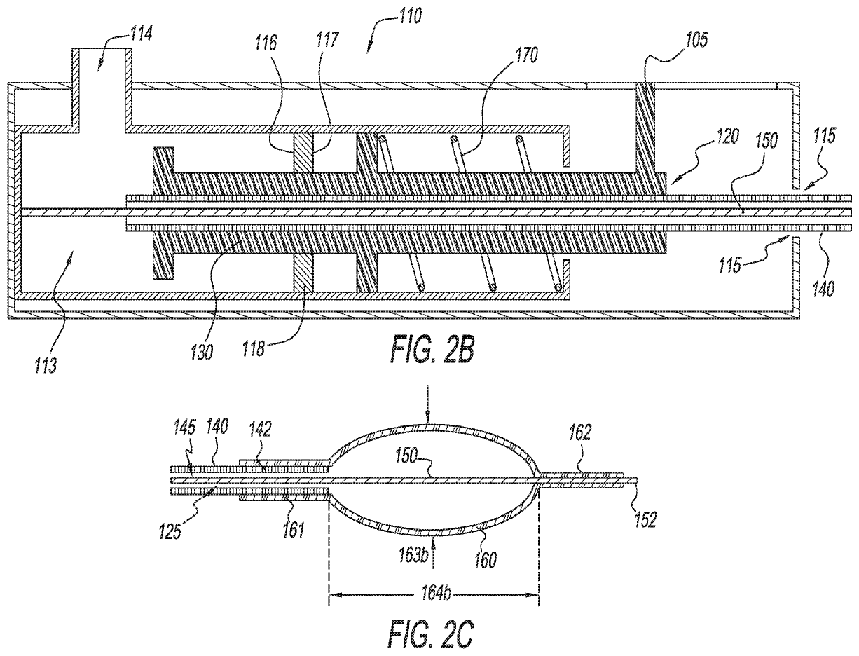

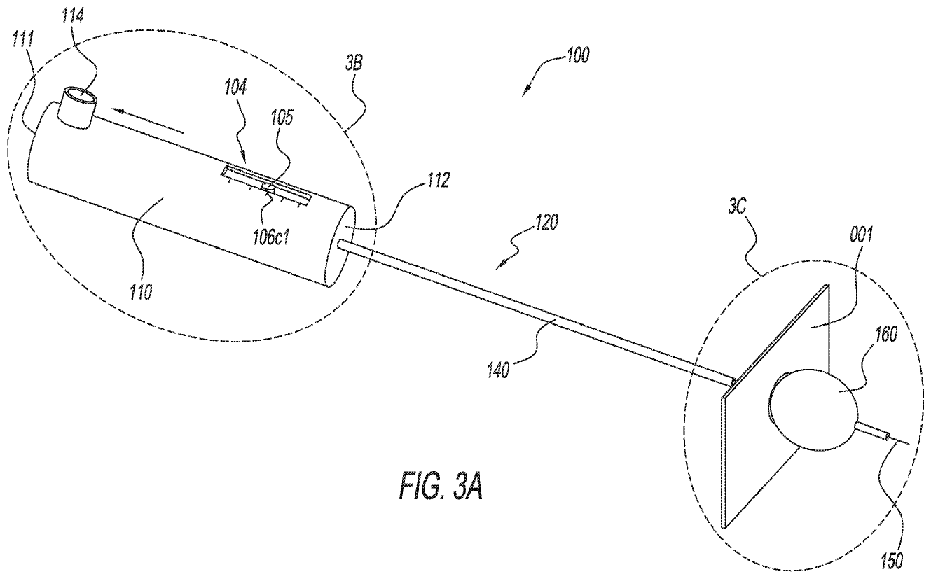

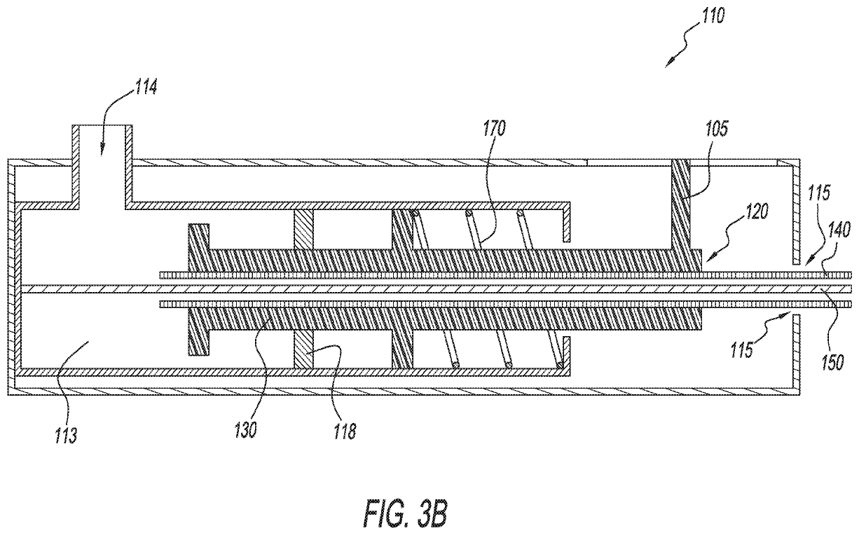

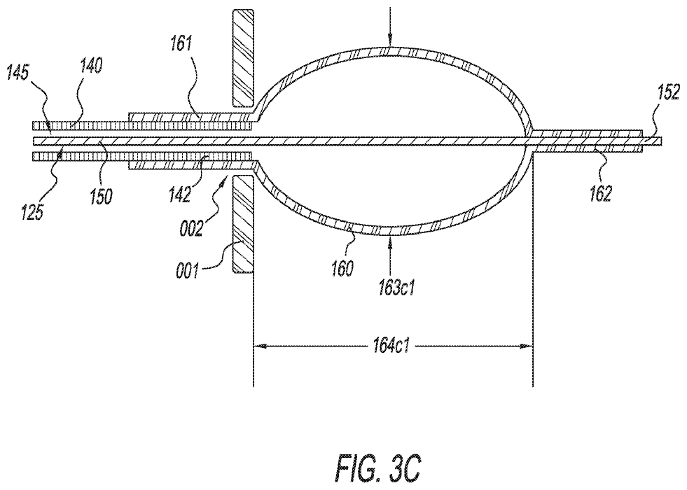

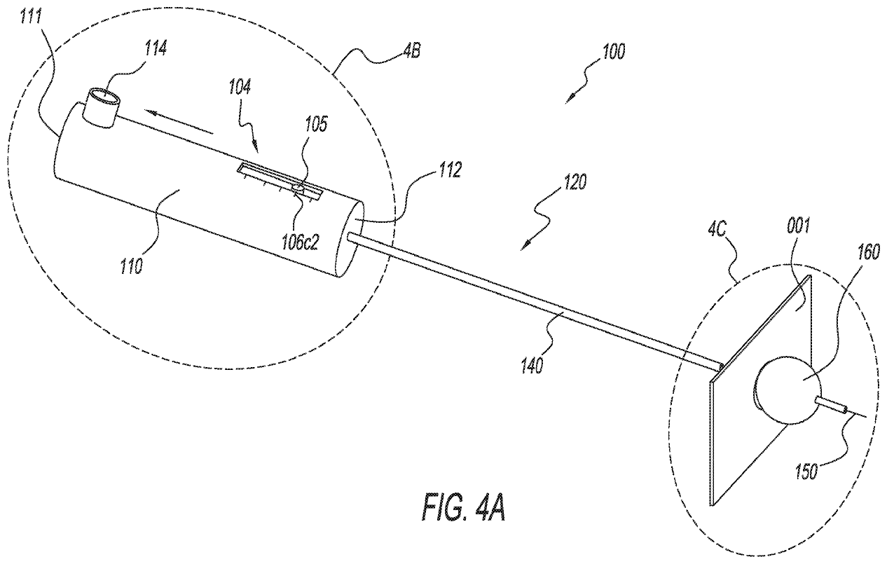

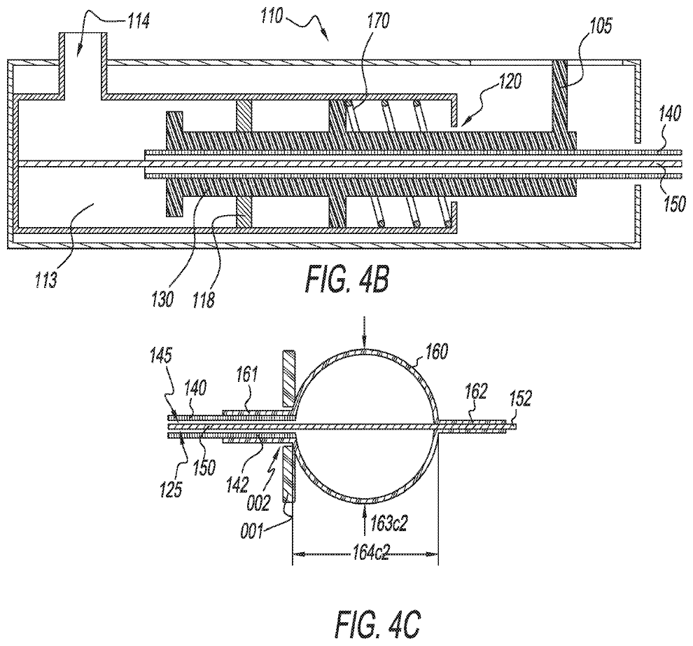

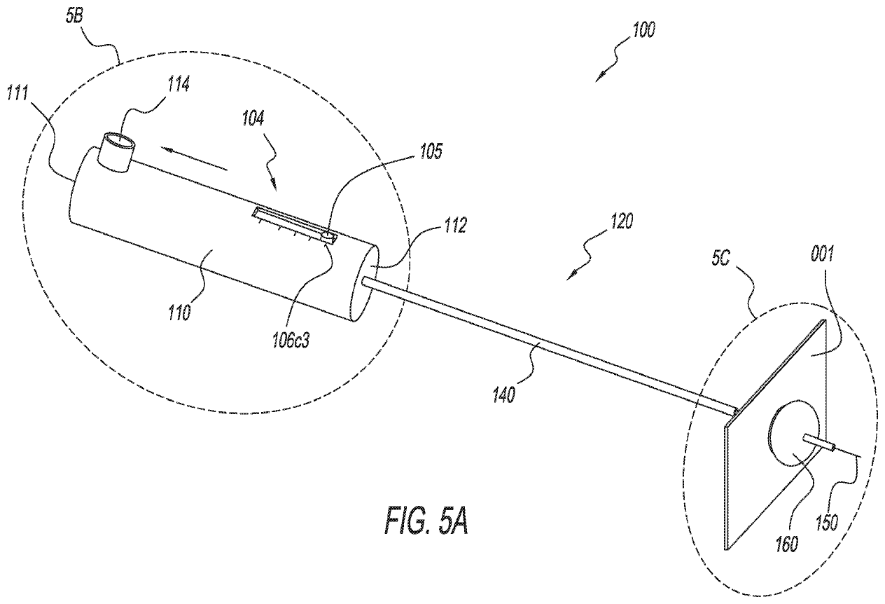

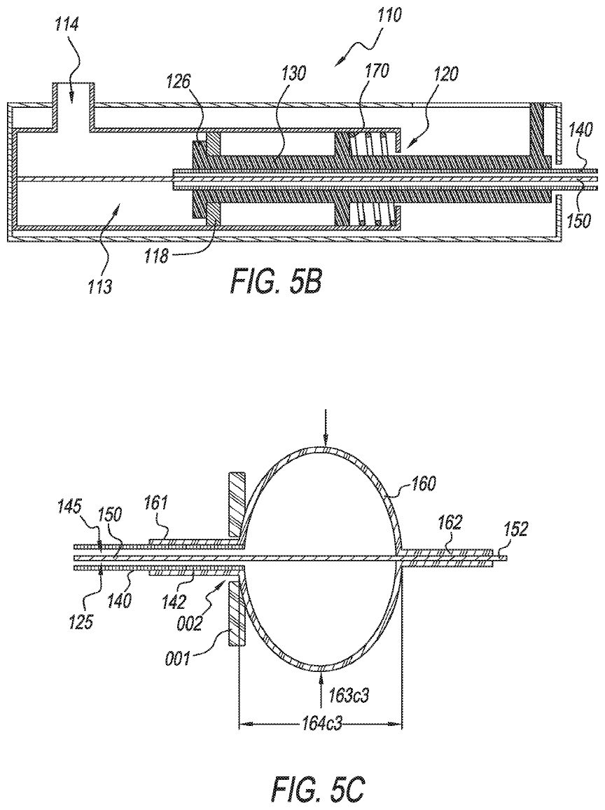

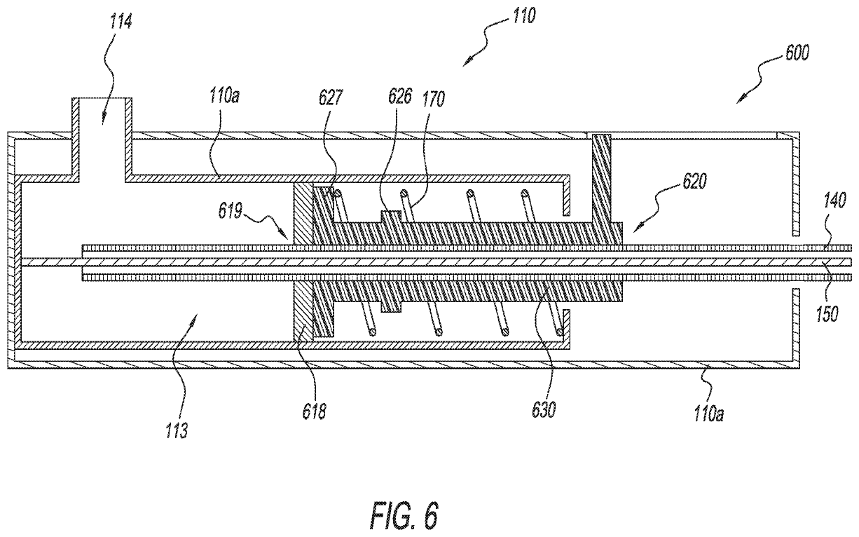

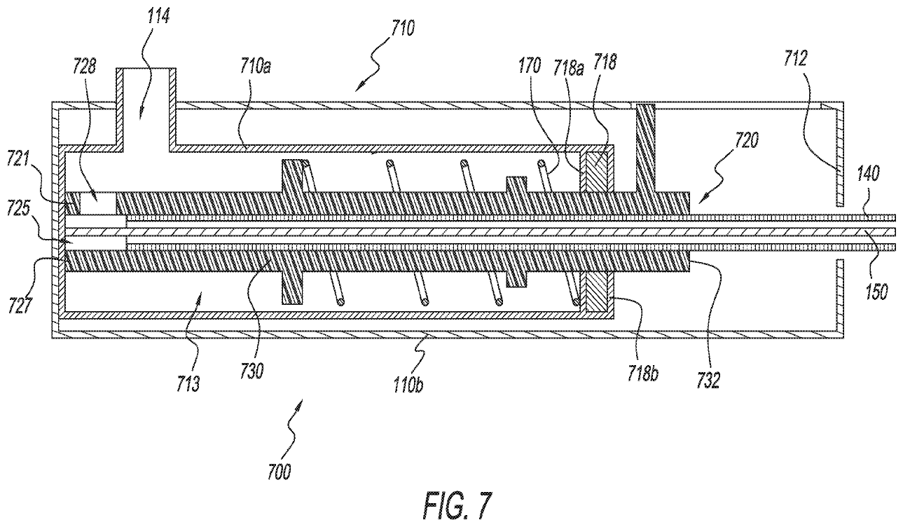

An example embodiment of an arteriotomy positioning device 100 is shown in FIGS. 1A-5C and may include a handle 110. A catheter assembly 120 may extend from the handle 110. The catheter assembly 120 may include a plunger 130 and a catheter 140. A core wire 150 may extend from the handle 110 and through a lumen 125 of the catheter assembly 120. A spring 170 may be positioned in the handle 110. An expandable support 160 may be located near the distal end of the device 100, and may be connected to both the core wire 150 and the catheter assembly 120.

The arteriotomy positioning device 100 may include a handle 110, as shown in FIGS. 1A-5B, The handle 110 may extend longitudinally between a proximal end 111 and a distal end 112. The handle 110 may include an inner housing 110a and an outer housing 110b as shown in FIG. 1B. In one aspect, the inner housing 110a and outer housing 110b may be molded as a single component. Features described as being included in the handle 110 could be included in one or both of the inner housing 110a or the outer housing 110b. Features described as being included in the inner housing 110a could alternatively be included in the outer housing 110b, and vice versa. The handle 110 may also include other components or features not shown in the figures.

The interior of the handle 110 may have a fluid chamber 113. The fluid chamber 113 may be formed by the inner housing 110a. The handle 110 may include an inflation port 114 that is in communication with the fluid chamber 113. The fluid chamber 113 may be in communication with the interior of the expandable support 160 via a lumen 125 of the catheter assembly 120. Therefore, a syringe or other source of inflation fluid may be coupled to the inflation port 114 to provide inflation fluid to fluid chamber 113, such that the expandable support 160 is able to expand, as described below. The inflation fluid may be saline, air, or another fluid appropriate for inflation. A valve (not shown) may also be coupled to the inflation port 114 to selectively allow fluid to enter and exit the fluid chamber 113. These features may be particularly useful if the expandable support is an inflatable balloon.

The distal end 112 of the handle 110 may also include a distal port 115 aligned along a longitudinal axis 103 of the device 100. The distal port 115 may be in communication with the interior of the handle 110. The catheter assembly 120 may extend through the distal port 115, such that a proximal end of the catheter assembly 120 may be located on the interior of the handle 110, and a distal end of the catheter assembly 120 may be located exterior to the handle 110.

The interior of the handle 110 may also have a second chamber 109, as shown in FIG. 1B, The second chamber 109 may be an area inside the inner housing 110a, but outside the fluid chamber 113. The second chamber 109 may house the spring 170 and a portion of the catheter assembly 120. As shown in FIG. 1B, the second chamber 109 may house a portion of the plunger 130 and a portion of the catheter 140. The second chamber 109 may be distal to the fluid chamber 113, and they may be separated by a seal 118. In other embodiments, the spring 170 and/or plunger 130 may be positioned either inside the fluid chamber 113, or inside the handle 110 but outside both the fluid chamber 113 and the second chamber 109. In still other embodiments, the second chamber may be omitted, and the spring 170 and/or plunger 130 may be located in the fluid chamber 113 and/or inside the handle 110 but outside the fluid chamber 113.

The handle 110 may include a seal 118 (e.g., an o-ring) around an opening in a distal end of the fluid chamber 113. The seal 118 may separate the fluid chamber 113 from the rest of the interior of the handle 110. The seal 118 may have a channel 119 that allows fluid to enter and exit the fluid chamber 113. The channel 119 in the seal 118 may be aligned along a longitudinal axis 103 of the device 100. The seal 118 may be substantially fixed relative to the handle 110. The handle 110 may further have proximal and/or distal seal retaining walls (similar to those shown in FIG. 7 as 718a and 718b, respectively) to prevent the seal 118 from moving within fluid chamber 113, Alternatively, the seal 118 may be connected to the handle 110 using an adhesive, or the seal 118 may be inserted into a groove in the handle 110, or it can be held in place relative to the handle 110 by any other appropriate mechanism.

The arteriotomy positioning device 100 may further include a core wire 150, The core wire 150 may be an elongate member having a proximal end 151 and a distal end 152. The core wire 150 is shown as being solid in all embodiments; however, any of the embodiments could alternatively use a core wire with one or more lumens.

The core wire 150, and preferably the proximal end 151 of the core wire 150, may be connected to the handle 110. Movement of the handle 110 may also move the core wire 150, at least when the device 100 is in the locating and tension states as described below. In the embodiment of FIG. 1A, the core wire 150 is connected to the inner housing 110a, and more precisely, the proximal end 151 of the core wire 150 is connected to the proximal end of the fluid chamber 113. The core wire 150 may extend substantially along the longitudinal axis 103 of the device 100, The core wire 150 may extend through at least one, and preferably both, of the channel 119 in the seal 118 and the distal port 115 of the handle 110.

The arteriotomy positioning device 100 may further include a catheter 140. The catheter 140 may be an elongate tubular member having a proximal end 141 and a distal end 142, A lumen 145 may extend from the proximal end 141 to the distal end 142. Thus, the catheter 140 may have an outer surface 143 and an inner surface 144.

The arteriotomy positioning device 100 may further include a plunger 130. The plunger 130 may have a proximal end 131 and a distal end 132. The plunger 130 may have a lumen 135, which may extend from the proximal end 131 to the distal end 132. Thus, the plunger 130 may have an outer surface 133 and an inner surface 134.

The catheter 140 may be connected to the plunger 130 to form a catheter assembly 120. The position of the catheter 140 relative to the plunger 130 may be substantially fixed, such that the catheter 140 moves together with the plunger 130, i.e., the catheter 140 is moveable upon movement of the plunger 130. The catheter 140 may be inserted into the lumen 135 of the plunger 130, such that the outer surface 143 of the catheter 140 may be in contact with, and preferably connected to, the inner surface 134 of the plunger 130.

The catheter assembly 120 may have a lumen 125. The lumen 125 may be formed by one or more of the lumen 145 of the catheter 140 and the lumen 135 of the plunger 130. As shown in FIGS. 1B-5B, the catheter 140 may extend along the entire lumen 135 of the plunger 130, such that the lumen 125 of the catheter assembly 120 is the lumen 145 of the catheter 140. Alternatively, the catheter 140 may extend along part of the lumen 135 of the plunger 130, such that the lumen 125 of the catheter assembly 120 is formed by both the lumen 145 of the catheter 140 and the lumen 135 of the plunger 130.

The catheter assembly 120 may have a proximal end 121 and a distal end 122. Preferably, the distal end 142 of the catheter 140 forms the distal end 122 of the catheter assembly 120. The proximal end 141 of the catheter 140, the proximal end 131 of the plunger 130, or both may form the proximal end 121 of the catheter assembly 120.

In other embodiments, the plunger 130 and the catheter 140 may be formed as a single part. In still other embodiments, the plunger 130 and the catheter 140 may be separate parts connected to one another using an adhesive, interference fit, or any other appropriate attachment mechanism known in the art. In some embodiments, features described as being included on the plunger 130 (including, but not limited, to the tension stop 126, resting stop 127, and/or various features of the device status indicator 104 (to be discussed below) may alternatively be included on the catheter 140.

The catheter assembly 120 may extend along the longitudinal axis 103 of the device 100, and may be positioned partially inside the handle 110. The proximal end 121 of the catheter assembly 120 may be positioned inside the handle 110, and preferably inside the fluid chamber 113. The lumen 125 of the catheter assembly 120 may be in communication with the fluid chamber 113 and inflation port 114 of the handle 110. The core wire 150 may extend through the lumen 125 of the catheter assembly 120. The distal end 152 of the core wire 150 may be distal to the distal end 122 of the catheter assembly 120.

The catheter assembly 120 may slidably extend through one or both of the channel 119 in the seal 118 and the distal port 115 of the handle 110. The catheter assembly 120 may sealingly engage the seal 118 of the handle 110, such that fluid leaving the fluid chamber 113 through the channel 119 in the seal 118 will flow through the lumen 125 of the catheter assembly 120. If the plunger 130 extends through the seal 118, as shown in FIGS. 1B-5B, the plunger 130 may form a seal with the seal 118 of the handle 110. Alternatively, if the proximal and distal ends 131, 132 of the plunger 130 are both either inside the fluid chamber 113 or outside the fluid chamber 113 (as shown in FIG. 6), then the catheter 140 may extend through and form a seal with the seal 118 of the handle 110. Therefore, when the catheter assembly 120 engages the seal 118 of the handle 110, fluid may enter and/or exit the fluid chamber 113 through the lumen 125 of the catheter assembly 120 and the inflation port 114 of the handle 110.

The device 100 may include two sets of stops: a resting stop and a tension stop. As the catheter assembly 120 moves proximally relative to the handle 110 and core wire 150, a resting stop 127 on the catheter assembly 120 may contact a corresponding resting stop 117 of the handle 110, thereby preventing further proximal movement of the catheter assembly 120. As the handle 110 and core wire 150 move proximally relative to the catheter assembly 120, a tension stop 126 on the catheter assembly 120 may contact a corresponding tension stop 116 of the handle 110, thereby preventing further proximal movement of the handle 110 and core wire 150.

In the embodiment of FIGS. 1B and 5B, the resting stop 117 of the handle 110 may be a distal surface of seal 118 (or a distal seal retaining wall, not shown), and the tension stop 116 of the handle 110 may be a proximal surface of the seal 118 (or a proximal seal retaining wall, not shown). Alternatively, the tension and resting stops of the handle 110 may be formed by other walls and/or features of the handle 110 based on the configuration of the handle 110 and the catheter assembly 120. For example, the proximal wall of the fluid chamber 113 may form a resting stop. A distal wall of the fluid chamber 113, or a wall at the distal end 112 of the handle 110, may form a tension stop. Various other features not shown in the figures may also be included in the handle to act as tension and resting stops.

The tension and resting stops 126, 127 on the catheter assembly 120 may preferably be included in the plunger 130. The stops 126, 127 may be projections extending from the outer surface 133 of the plunger 130, as shown in FIGS. 1B and 5B. Alternatively, the proximal end 121 of the catheter assembly 120 may form the resting stop, and/or the distal end 132 of the plunger 130 may form the tension stop. Based on the configuration of the handle 110 and the catheter assembly 120, various other features not shown in the figures may also be included in the catheter assembly to act as tension and resting stops.

The catheter assembly 120 may be positioned such that the tension stop 126 and the resting stop 127 of the catheter assembly 120 are inside the handle 110, although their positions within the handle 110 depend on the configuration of the catheter assembly 120 and the handle 110. In the embodiment shown in FIGS. 1B-5B, the tension stop 126 may be positioned inside the fluid chamber 113 of the handle 110. The resting stop 127 may be positioned outside the fluid chamber 113 but inside the handle 110, and as shown in FIGS. 1B-5B, the resting stop 127 may be positioned inside the second chamber 109). Alternatively, the resting stop may be positioned inside the fluid chamber 113 of the handle 110, while the tension stop may be positioned outside the fluid chamber 113 but inside the handle 110. The tension stop and the resting stop may also both be inside the fluid chamber 113 of the handle 110 (as shown in FIG. 7), or may both be outside the fluid chamber 113 but inside the handle 110 (not shown).

In the embodiment shown in FIG. 1B, the resting stops 117, 127 on the handle 110 and catheter assembly 120 are positioned distally relative to their respective tension stops 116, 126. Depending on the configuration of the plunger 130 and handle 110, the resting stops on the handle 110 and catheter assembly 120 may alternatively be positioned proximally relative to their respective tension stops on the handle 110 and catheter assembly 120.

The catheter assembly 120 may be moveable relative to the handle 110 and the core wire 150. Preferably, the catheter assembly 120 is slidable relative to the handle 110 and the core wire 150 along a longitudinal axis 103 of the device 100. The catheter assembly 120 may be able to slide through one or both of the channel 119 in the seal 118 or the distal port 115 of the handle 110.

The arteriotomy positioning device 100 may further include an expandable support 160. The expandable support 160 may be a balloon, a group of splines, a combination thereof, or any expandable structure that can move between a low-profile configuration, a medium-profile configuration, and a high-profile configuration. The expandable support 160 may have a proximal bonding region 161 at its proximal end and a distal bonding region 162 at its distal end. The axial length (also referred to as the length) of the expandable support 160 may be the distance between the proximal bonding region 161 and the distal bonding region 162, as measured along the longitudinal axis 103 of the device 100. A width of the expandable support 160 may be measured substantially perpendicularly to the longitudinal axis 103 of the device 100, at the widest part of the expandable support 160. If the expandable support 160 is a balloon, the balloon may be provided in standard shapes and sizes, and may be made from conventional materials. If the expandable support is in the shape of a sphere or ellipsoid, the width would be the maximum diameter of the expandable support 160 in a direction substantially perpendicularly to the longitudinal axis 103 of the device 100. As will be described below, the length and the width of the expandable support 160 may change as the expandable support 160 is moved between the low-profile configuration, the medium-profile configuration, and the high-profile configuration.

The expandable support 160 may be connected to the catheter 140 and the core wire 150. The proximal bonding region 161 of the expandable support may be connected to the distal end 122 of the catheter assembly 120. More specifically, the proximal bonding region 161 of the expandable support 160 may be connected to the distal end 142 of the catheter 140. Therefore, the proximal bonding region 161 of the expandable support 160 may be substantially fixed relative to the catheter assembly 120, but may be moveable relative to the core wire 150 and handle 110. The distal bonding region 162 of the expandable support 160 may be connected to the distal end 152 of the core wire 150. Therefore, the distal bonding region 162 of the expandable support 160 may be substantially fixed relative to the core wire 150 and handle 110, but moveable relative to the catheter assembly 120. If the expandable support 160 is a balloon, the lumen 145 of the catheter 140 may communicate with the interior of the balloon. Therefore, the interior of the balloon may be in communication with the inflation port 114 via the lumen 125 of the catheter assembly 120 and the fluid chamber 113 of the handle 110.

The length of the expandable support 160 may change if the catheter assembly 120 is moved relative to the core wire 150, allowing the expandable support 160 to move between the low-profile configuration, the medium-profile configuration, and the high-profile configuration. In the low-profile configuration, the expandable support 160 may have a first length 164a and a first width 163a as shown in FIG. 1C. In the medium-profile configuration, the expandable support 160 may have a second length 164b and a second width 163b as shown in FIG. 2C. In the high-profile configuration, the expandable support 160 may have a third length 164c1, 164c2, or 164c3 and a third width 163c1, 163c2, or 163c3 as shown in FIGS. 3C, 4C, and 5C, respectively. Generally, the length may decrease and the width may increase as the expandable support 160 moves from the low-profile configuration to the medium-profile configuration to the high-profile configuration. The first length 164a may be longer than the second length 164b, both of which may be longer than the third lengths 164c1, 164c2 and 164c3. The first width 163a may be smaller than the second width 163b, both of which may be smaller than the third width 163c1, 163c2, and 163c3.

For simplicity, the first width 163a, first length 164a, second width 163b, second length 164b, third width 163c1, 163c2, and 163c3, and third length 164c1, 164c2 and 164c3 are each referred to as a “width” or a “length”, but each width may encompass a range of widths and each length may encompass a range of lengths. For example, FIGS. 30, 4C, and 50 show that the third width may include widths 163c1, 163c2, and 163c3 (listed from narrowest to widest), and the third length may include lengths 164c1, 164c2, and 164c3 (listed from longest to shortest). Each of widths 163c1, 163c2, and 163c3 may be wider than any first width 163a and second width 163b, and each of lengths 164c1, 164c2 and 164c3 may be shorter than any first length 164a or second length 164b.

The arteriotomy positioning device 100 may also include a spring 170. The spring 170 may apply a proximal force to the catheter assembly 120 to bias the catheter assembly 120 proximally relative to the handle 110 and the core wire 150, and similarly, to bias the handle 110 and the core wire 150 distally relative to the catheter assembly 120. The spring 170 may be positioned inside the handle 110, and may be outside the fluid chamber 113 (as shown in FIG. 1B), or, alternatively, inside the fluid chamber 113 (as shown in FIG. 7). The spring 170 may extend between a spring-contacting surface of the handle 110 and a spring-contacting surface of the catheter assembly 120. The locations of the spring-contacting surfaces may vary based on the configuration of the handle 110 and catheter assembly 120. Preferably, if spring 170 is a compression spring, the spring-contacting surface of the handle 110 may be distal to a spring-contacting surface of the catheter assembly 120. The spring-contacting surfaces may be a proximal-facing surface of the handle 110 and a distal-facing surface of the catheter assembly 120. For example, in the embodiment shown in FIG. 1B, the spring-contacting surfaces are a distal wall of the inner housing 110a and the resting stop 127 of the catheter assembly 120. However, various features of the handle 110 and the catheter assembly 120 may serve as spring-contacting surfaces.

The device 100 may be designed to prevent the user from unintentionally pulling the expandable support 160 through the blood vessel 001 when applying tension to the catheter assembly 120. The force required to compress the spring 170 and move the housing 110 and core wire 150 proximally relative to the catheter assembly 120 when applying tension to the catheter assembly 120 may preferably be less than the force required to pull the expandable support 160 through the blood vessel 001. The force required to pull the expandable support 160 through the blood vessel 001 may increase as the width 163 of the expandable support 160 increases. For example, the force to pull the expandable support 160 through the arteriotomy when the expandable support 160 is in the high-profile configuration may be higher than the force to pull the expandable support 160 through the arteriotomy when the expandable support is in the medium-profile configuration. Therefore, increasing the tension applied to the catheter assembly 120 may also increase the force required to pull the expandable support 160 through the blood vessel 001, allowing the user to apply an increasing amount of force without pulling the expandable support 160 through the vessel.

During use, the arteriotomy positioning device 100 may be moveable between at least three states: a resting state, a locating state, and a tension state.

When the arteriotomy positioning device 100 is in the resting state (see FIGS. 1A-1C), the expandable support 160 may be in the low-profile configuration, having a resting width (or first width) 163a and a resting length (or first length) 164a. The spring 170 may bias the catheter assembly 120 proximally to its proximal-most position relative to the core wire 150 and handle 110, thereby pulling the proximal end 161 of the expandable support 160 proximally which minimizes the resting width 163 of the expandable support 160. The resting stop 127 of the catheter assembly 120 may rest on the resting stop 117 of the handle 110. Therefore, when the device 100 is in the resting state, the resting stop 127 may prevent the catheter assembly 120 from moving proximally relative to the handle 110 and the core wire 150, and the spring 170 may resist distal movement of the catheter assembly 120 relative to the handle 110 and the core wire 150.

The device may include a releasable resting lock to maintain the expandable support 160 in the resting state. When engaged, the resting lock may prevent the catheter assembly 120 from moving distally relative to the handle 110 and the core wire 150, thereby preventing the expandable support 160 from moving from the low-profile configuration to the medium-profile configuration or high-profile configuration. When the resting lock is disengaged, the catheter assembly 120 may be able to move distally relative to the handle 110 and the core wire 150, allowing the expandable support 160 to move from the low-profile configuration to the medium-profile configuration or high-profile configuration. The user may be able to selectively engage and disengage the resting lock.

If the expandable support 160 is a balloon, the resting lock may be a valve associated with the inflation port 114. The resting lock may be engaged by closing the valve, thereby preventing inflation fluid from entering the fluid chamber 113 through the inflation port 114. The resting lock may be disengaged by opening the valve, thereby allowing inflation fluid to enter the fluid chamber 113 through the inflation port.

When the arteriotomy positioning device 100 is in the locating state (see FIGS. 2A-2C), the expandable support 160 may be in the medium-profile configuration, having a locating width (or second width) 163b and a locating length (or second length) 164b. The locating width 163b may be larger than the resting width 163a. The locating length 164b may be shorter than the resting length 164a. In the locating state, the catheter assembly 120 may be positioned distally relative to the core wire 150 and handle 110, as compared to their respective positions in the resting state. The distance between the distal end 122 of the catheter assembly 120 and the distal end 112 of the handle 110 may be increased in the locating state compared to the resting state. The distance between the distal end 122 of the catheter assembly 120 and the distal end 152 of the core wire 150 may be decreased in the locating state compared to the resting state. The distance between the distal end 152 of the core wire 150 and the distal end 112 of the handle 110 may be substantially constant between the resting state and locating state. The resting stop 127 of the catheter assembly 120 may be spaced from the resting stop 117 of the handle 110. The spring 170 may be more compressed in the locating state compared to the resting state. Therefore, the spring 170 may still resist distal movement of the catheter assembly 120 relative to the handle 110 and the core wire 150. If the expandable support 160 is a balloon, the balloon may be inflated when the device is in the locating state.

The device may include a releasable locating lock to prevent the device 100 from unintentionally moving from the locating state (or the tension state) back to the resting state. When engaged, the locating lock may limit proximal movement of the catheter assembly 120 relative to the handle 110 and the core wire 150 such that the expandable support 160 can move between the medium-profile configuration and high-profile configuration, but it cannot move to the low-profile configuration. When the locating lock is disengaged, the expandable support 160 may be able to move between the medium-profile configuration (or the high-profile configuration) and the low-profile configuration. The user may be able to selectively engage and disengage the locating lock.

If the expandable support 160 is a balloon, the locating lock may be a valve (not shown) associated with the inflation port 114. The same valve may act as the resting lock and the locating lock. The locating lock may be engaged by closing the valve after inflation fluid has entered the fluid chamber 113, thereby preventing fluid from exiting the fluid chamber 113 through the inflation port 114. The locating lock may be disengaged by opening the valve, thereby allowing inflation fluid to exit the fluid chamber 113 through the inflation port 114.

When the arteriotomy positioning device 100 is in the tension state (see FIGS. 3A-50), the expandable support 160 may be in the high-profile configuration, having a tension width (or third width) 163c1, 163c2, and 163c3 and a tension length (or third length) 164c1, 164c2, and 164c3. The tension width 163c1, 163c2, and 163c3 may be larger than both the resting width 163a and the locating width 163b. The tension length 164c1, 164c2, and 164c3 may be shorter than both the resting length 164a and the locating length 164b. In the tension state, the core wire 150 and handle 110 may be positioned proximally relative to the catheter assembly 120, as compared to their respective positions in both the resting state and the locating state. The distance between the distal end 122 of the catheter assembly 120 and the distal end 112 of the handle 110 may be increased in the tension state compared to the resting and locating states. The distance between the distal end 122 of the catheter assembly 120 and the distal end 152 of the core wire 150 may be decreased in the tension state compared to the resting and locating states. The distance between the distal end 152 of the core wire 150 and the distal end 112 of the handle 110 may be substantially constant between the resting, locating, and tension states. If the expandable support 160 is a balloon, the balloon may remain inflated when the device is in the tension state. The device may have substantially the same amount of inflation fluid in the tension state as it does in the locating state.

FIGS. 3C, 4C, and 5C show that the tension width and tension length may vary while the expandable support is in the high-profile configuration. This variation may be correlated to the amount of tension applied to the catheter assembly 120. For example, if an adequate amount of tension is applied to the catheter assembly 120, as shown in FIG. 4C, the expandable support may have a width 163c2 and length 164c2. If too much tension is applied to the catheter assembly 120, as shown in FIG. 5C, the expandable support 160 may have a width 163c3 that is greater than width 163c2, and a length 164c3 that is shorter than length 164c2. If too little tension is applied to the catheter assembly 120, as shown in FIG. 3C, the expandable support 160 may have a width 163c1 that is less than width 163c2, and a length 164c1 that is longer than length 164c2.

When the arteriotomy positioning device 100 is in the tension state, the resting stop 127 of the catheter assembly 120 may be spaced from a resting stop 117 of the handle 110. The tension stop 126 of the catheter assembly is not necessarily in contact with the tension stop 116 on the handle 110. However, increasing the tension on the catheter assembly 120 to an undesirably high level may cause the tension stop 126 to contact a tension stop 116 of the handle 110, preventing further proximal movement of the core wire 150 and handle 110 relative to the catheter assembly 120, which limits the amount of tension on the catheter assembly 120. The spring 170 may be more compressed in the tension state compared to the resting and locating states. Therefore, the spring 170 may still resist proximal movement of the handle 110 and the core wire 150 relative to the catheter assembly 120, even if the tension stops 126, 116 on the catheter assembly and handle 110 are not in contact.

The arteriotomy positioning device 100 may include a device status indicator 104 which visually indicates both the status of the device 100 (i.e., whether the device is in the resting state, locating state, or tension state) and the amount of tension, if any, being applied to the catheter assembly 120. The device status indicator 104 may allow the user to compare a feature on the catheter assembly 120 to a feature on the handle 110 to determine the longitudinal position of the catheter assembly 120 relative to the core wire 150.

The device status indicator 104 may include an indicator feature 105 on one component that moves relative to a series of markings 106 (including but not limited to lines, colored bands, raised or lowered features, etc.) on another component. The device status indicator 104 may include a series of markings 106 on the handle 110 and an indicator feature 105 on the catheter assembly 120. For example, the indicator feature 105 may be a protrusion on the plunger 130, and the markings 106 may be on the handle 110 as shown in FIG. 1A. Alternatively, the device status indicator may include a series of markings on the catheter assembly, and an indicator feature on the handle. For example, the indicator feature may be a window in the handle as shown in FIG. 8, or it could simply be the distal port of the handle as shown in FIG. 11. In any case, when the catheter assembly 120 slides relative to the handle 110, the indicator feature 105 aligns with one of the markings 106 to show the status of the device 100, and the amount of tension, if any, being applied to the catheter assembly 120. The handle 110 may include an indicator window 107 to allow the user to visualize either a marking 106 on the catheter assembly 120 or a position of an indicator feature 105 on the catheter assembly relative to markings 106 on the handle 110 near the indicator window 107.

The device status indicator 104 may include the following markings 106: 1) a marking 106a indicating that the device 100 is in the resting state, the expandable support 160 is in a low-profile configuration, and the catheter assembly 120 is not under tension, 2) a marking 106b indicating that the device 100 is in the locating state and the expandable support 160 is in a medium-profile configuration, and the catheter assembly 120 is not under tension, and 3) one or more markings 106c1, 106c2, 106c3 indicating that the device 100 is in the tension state, the expandable support 160 is in a high-profile configuration, and the catheter assembly 120 is under tension. Marking 106c1 may indicate that the catheter assembly 120 is under tension with too little force, marking 106c2 may indicate that the catheter assembly 120 is under tension with appropriate force, and marking 106c3 may indicate that the catheter assembly 120 is under tension with too much force. If the markings 106 are included on the catheter assembly 120, marking 106a may preferably be the distal-most marking, followed in order by marking 106b, then marking 106c1, then marking 106c2, and finally, marking 106c3, which may be the proximal-most marking. If the markings are included on the handle 110, marking 106a may preferably be the proximal-most marking, followed in order by marking 106b, then marking 106c1, then marking 106c2, and finally, marking 106c3, which may be the distal-most marking. However, the reverse order may also be used, or the markings may be arranged in a different order depending on the configuration of the device 100 and the device status indicator 104. One or more of markings 106a, 106b, 106c1, 106c2, and 106c3 may be omitted, or additional markings may be included.

The following exemplary method (described in detail in the following paragraphs) may be used when locating an arteriotomy and performing a procedure. The method may include steps of inserting the expandable support through the arteriotomy and into the vessel, moving the device to the locating state, withdrawing the handle proximally until the expandable support contacts the arteriotomy, continuing to withdraw the handle proximally to apply tension to the catheter assembly and move the device to the tension state, performing a procedure, returning the device to its resting state, and withdrawing the device from the patient. It is understood that one or more of these steps may be omitted and other steps may be included in this method.

First, the user may insert the device 100 into the patient, thereby inserting the expandable support into the vessel 001 through the arteriotomy 002, as illustrated in FIG. 1A. During insertion, the arteriotomy positioning device 100 may be in the resting state, and the device status indicator 104 may be in a position that indicates that the device 100 is in the resting state. A resting lock may be engaged to secure the device 100 in the resting state during insertion. The expandable support 160, the distal end 122 of the catheter assembly 120 (preferably the distal end 142 of the catheter 140), and the distal end 152 of the core wire 150 may be inserted into the vessel 001 through the arteriotomy 002, as shown by the arrow in FIG. 1A. The expandable support 160 may be in the low-profile configuration. The resting width 163a of the expandable support may be smaller than the width of the arteriotomy 002, which allows the expandable support to fit through the arteriotomy 002. The handle 110, core wire 150, and catheter assembly 120 may all move together during insertion, since a distal force is not being applied to the distal end 142 of the catheter 140. When the device 100 has been inserted and the expandable support 160 is positioned in the vessel 001, a resting lock may be disengaged, allowing the device 100 to be moved from the resting state to the locating state.

When the expandable support 160 is positioned inside the vessel, the user may move the arteriotomy positioning device 100 from the resting state to the locating state. The expandable support 160 may move from the low-profile configuration to the medium-profile configuration, while the catheter assembly 120 may move distally relative to the handle 110 and the core wire 150, The width 163 of the expandable support 160 may increase from the resting width 163a to the locating width 163b. The locating width 163b may be larger than the width of the arteriotomy 002 to prevent the expandable support from being pulled through the arteriotomy 002 while the device is in the locating state. The length 164 of the expandable support 160 may decrease from the resting length 164a to the locating length 164b. The distal resting stop 127 of the catheter assembly 120 may move away from the resting stop 117 of the handle 110, such that they are no longer in contact. The spring 170 may still resist distal movement of the catheter assembly 120 relative to the handle 110 and the core wire 150. The spring 170 may compress when the device 100 is moved from the resting state to the locating state. The device status indicator 104 may move from a position indicating that the device 100 is in the resting state to a position indicating that the device 100 is in the locating state. Once the device 100 reaches the locating state, a locating lock may be engaged to prevent the device 100 from unintentionally reverting to the resting state during use.

If the expandable support 160 is a balloon, the step of moving the arteriotomy positioning device 100 from the resting state to the locating state may involve inflating the balloon. The device 100 may be moved from the resting state to the locating state by pushing the inflation fluid through the inflation port 114, into the fluid chamber 113 of the handle 110, through the lumen 125 of the catheter assembly 120, and into the interior of the balloon. Once the device 100 reaches the locating state, a valve (not shown) associated with the inflation port 114 may be closed to prevent the inflation fluid from flowing out of the fluid chamber 113 through the inflation port 114.

When the arteriotomy positioning device 100 is in the locating state, the user may withdraw the handle 110 proximally to move the expandable support 160 closer to the arteriotomy 002, as shown by the arrow in FIG. 2A. The catheter assembly 120 is not under tension at this point because no distal force is being applied to the distal end 142 of the catheter 140. Therefore, the handle 110, core wire 150, and catheter assembly 120 may all move proximally together, and the device 100 may remain in the locating state until the user locates the arteriotomy 002 (in other words, until the expandable support 160 contacts the vessel 001 at the arteriotomy 002). The device status indicator 104 may show that a small amount of tension is being applied to the catheter assembly 120 to indicate that the expandable support 160 has contacted the vessel 001 and the device 100 is in the correct position.

When the expandable support 160 contacts the arteriotomy 002, the user may continue withdrawing the handle 110 proximally (as shown by the arrows in FIGS. 3A, 4A, and 5A) to apply tension to the catheter assembly 120 and bring the device 100 to the tension state. When the device 100 is under tension, the catheter assembly 120 is constrained by the vessel wall 001 and remains substantially stationary. Applying a proximal force to the handle 110 causes the vessel wall 001 to apply a distal force to the expandable support 160, and this distal force is transmitted to the distal end 122 of the catheter assembly 120 (and preferably to the distal end 142 of the catheter 140). Meanwhile, the user applies a proximal force to the handle 110, which compresses the spring 170 and transmits the proximal force to the proximal end 121 of the catheter assembly 120 (and preferably to the plunger 130). Tension may be created by the distal force applied near the distal end 122 of the catheter assembly 120 and the proximal force applied near the proximal end 121 of the catheter assembly 120. When the device is moved from the locating state to the tension state, the width 163 of the expandable support 160 may increase from the locating width 163b to the tension width 163c, and the length 164 of the expandable support 160 may decrease from the locating length 164b to the tension length 164c. The device status indicator 104 may indicate whether the appropriate amount of tension is being applied. If the device status indicator 104 shows that too much or too little tension is being applied to the catheter assembly 120, the user may decrease or increase (respectively) the proximal force applied to the handle 110 and/or core wire 150 until the device status indicator 104 shows that an appropriate amount of tension is being applied to the catheter assembly 120. Contacting a tension stop 126 on the catheter assembly 120 with a tension stop 116 on the handle 110 may also prevent the user from applying too much tension to the catheter assembly 120. A locating lock may remain engaged as the device moves from the locating state to the tension state in order to prevent the device from moving to the resting state.

If the expandable support 160 is a balloon, the balloon may remain inflated as the arteriotomy positioning device 100 is moved between the locating state and the tension state. Although shape of the balloon may change as the device 100 is moved between the locating state and the tension state, the amount of inflation fluid in the device may remain substantially constant. Therefore, a valve associated with the inflation port may remain closed as the device is moved between the locating state and the tension state.

When the device status indicator 104 shows that an appropriate amount of tension is being applied to the catheter assembly 120, the user can perform the intended procedure (e.g., applying a sealant or other closure device, in the case of a vascular closure procedure). The proximal force applied to the handle 110 and/or core wire 150 may be maintained throughout at least a portion of the procedure, such that the device status indicator 104 continues to show that an appropriate amount of tension is being applied to the catheter assembly 120.

After completion of the procedure, the user may return the arteriotomy positioning device 100 to its resting state. The proximal force applied to the handle 110 may be decreased, thereby releasing the tension on the catheter 140, allowing the catheter assembly 120 to move proximally relative to the handle 110 and the core wire 150, and returning the device to the locating state. A locating lock may be disengaged, and the catheter assembly 120 may be moved further proximally relative to the handle 110 and the core wire 150 to return the device 100 to its resting state. If the expandable support 160 is a balloon, the locating lock may be disengaged by opening a valve associated with the inflation port 114, which allows inflation fluid to exit the fluid chamber 113 through the inflation port 114 on the handle 110, causing the balloon to deflate and the device 100 to return to its resting state.

When the arteriotomy positioning device 100 has been returned to its resting state, the expandable support 160 may have the resting width 163a smaller than the width of the arteriotomy 002. Therefore, the expandable support may fit through the arteriotomy 002 and the device 100 may be removed from the patient. A proximal force may be applied to the handle 110, and the entire arteriotomy positioning device 100 may be withdrawn from the patient.

Various other components may be provided within the arteriotomy positioning device 100. In addition, the arteriotomy positioning device 100 may also be incorporated into other devices used in procedures that require access to a patient's vasculature. For example, the arteriotomy positioning device 100 may be incorporated into a vascular closure device. The vascular closure device may further incorporate features including a sealant, a pusher member, a protective sleeve, and various other components. The sealant may be positioned near the distal end of the catheter assembly, proximal to the expandable member. The pusher member may be positioned proximal to the sealant to prevent proximal movement of the sealant and/or tamp the sealant. The sealant and pusher member may be provided inside a protective sleeve, which may be withdrawn to deploy the sealant. Incorporating the arteriotomy positioning devices discussed above into a vascular closure device may help ensure that the sealant is positioned correctly (outside the blood vessel, but near the arteriotomy) before the user deploys and/or tamps the sealant. The device may further include additional components, actuators, and/or safety mechanisms for controlling the device while exposing the sealant, tamping the sealant, withdrawing the cartridge assembly and expandable support relative to the sealant and/or the protective sleeve.

A number of alternative embodiments of arteriotomy positioning devices are envisioned, including those shown in FIGS. 6-11. Several of the alternative embodiments may have one or more features in common. Other embodiments may have a handle, a catheter assembly, a core wire, and an expandable member. The core wire may be substantially fixed relative to the handle, at least while the device is in the locating and/or tension states. The catheter assembly may include a plunger substantially fixed relative to a catheter. The catheter assembly may be moveable relative to the handle and the core wire. The proximal end of the expandable support may be connected to the catheter, and the distal end of the expandable support may be connected to the core wire. The distance between distal end of the expandable support and a distal end of the handle may be substantially fixed, at least while the device is in the locating and/or tension states, whereas the proximal end of the expandable support may be moveable relative to the handle. The proximal end of the expandable support may be biased proximally, but moveable distally relative to the handle in response to various forces applied to the device. Applying tension to the catheter assembly may increase the width of the expandable support. The device may also have a device status indicator capable of indicating both the configuration of the expandable support and an amount of tension being applied to the catheter assembly. The exemplary alternative embodiments shown in FIGS. 6-11 show different configurations of arteriotomy positioning devices. It is understood that one of ordinary skill in the art would understand that one or more features from one embodiment may be combined with other features from other embodiments.

FIG. 6 shows an alternative embodiment of an arteriotomy positioning device 600, wherein the plunger 630 does not extend into the fluid chamber 113. Device 600 is similar to device 100, except for the catheter assembly 620 (specifically, the plunger 630) and the seal 618 (specifically, the channel 619 in the seal 618) may differ from catheter assembly 120 and seal 118. FIG. 6 shows an embodiment with the plunger 630 positioned entirely outside the fluid chamber 113 of the handle 110. Both the resting stop 627 and the tension stop 626 of the catheter assembly 620 may be positioned outside the fluid chamber 113. The seal 618 may form a resting stop on the handle 110 to limit proximal movement of the catheter assembly 620. A surface of the handle 110 (in FIG. 6, this surface may be a distal surface of the inner housing 110a) may form a tension stop on the handle 110 to limit proximal movement of the core wire 150 and/or housing 110 relative to the catheter assembly 620. The channel 619 in the seal 618 may have a smaller diameter in order to seal to the catheter 140, since the plunger 630 does not extend through the channel 619 in the seal 618.

FIG. 7 shows another alternative embodiment of an arteriotomy positioning device 700 wherein the handle 110 includes a fluid chamber 713, but not a second chamber. Device 700 is similar to device 100, except the handle 710 (specifically, the inner housing 710a) and the catheter assembly 720 (specifically, the plunger 730) may differ from the handle 100 and catheter assembly 120 of device 100. The spring 170 is shown as being positioned in the fluid chamber 713 in FIG. 7; however, it could also be positioned outside the fluid chamber 713, but within the handle 710. For example, a spring may be inserted over the catheter 140, such that the distal end of the spring 170 may contact a surface on the handle 710 (possibly the distal end 712 of the handle 710), and the proximal end of the spring may contact a surface on the plunger 730 (possibly the distal end 732 of the plunger 730). Additionally, the handle 710 of FIG. 7 shows proximal and distal seal retaining walls 718a, 718b which are one way in which the seal 718 may be secured in the handle. These seal retaining walls may be applied to various other embodiments in order to maintain the position of the seal relative to the handle.

Another unique feature of FIG. 7 is the fact that the proximal end 721 of the catheter assembly 720 forms the resting stop 727. The catheter assembly 720 may include a transverse channel 728 that communicates with the lumen 725 of the catheter assembly 720, as shown in FIG. 7. The transverse channel 728 may allow fluid to move from the inflation port 114 and into the lumen 725 of the catheter assembly 720 when the device 700 is in the resting position, as shown in FIG. 7. This concept could also be applied to embodiments with both a fluid chamber and a second chamber. Alternatively, the plunger could be designed such that the resting stop of the plunger may be located outside the fluid chamber 713, such that the resting stop may rest on the distal-most surface of the inner housing 710a when the device 700 is in the resting position.

FIG. 8 shows still another alternative embodiment of an arteriotomy positioning device 800 having an alternative device status indicator. Device 800 is similar to device 100, although the handle 810 (specifically, the outer housing 810b) and catheter assembly 820 (specifically, the plunger 830) may differ from handle 110 and catheter assembly 120. The indicator feature 805 is on the handle 810 and the markings 806 are on the catheter assembly 820. The indicator feature 805 may be a window in the handle 110. The window may be sized to show and/or align with one of the markings 806 at a time, thereby allowing the user to determine the status of the device as the markings 806 (shown as raised features in FIG. 8) on the catheter assembly 820 slide past the window on the handle 810.

FIG. 9 shows yet another exemplary embodiment of an arteriotomy positioning device 900. Device 900 is similar to device 100, except that the handle 910 and catheter assembly 920 (specifically, the plunger 930) may differ from handle 110 and catheter assembly 120. In this embodiment, the indicator feature 905 also functions as a resting stop. The spring 170 is located inside the handle 910, but outside the fluid chamber 113. The spring-contacting surfaces may be a distal end 932 of the plunger 930 and a distal end 912 of the outer housing 910b.

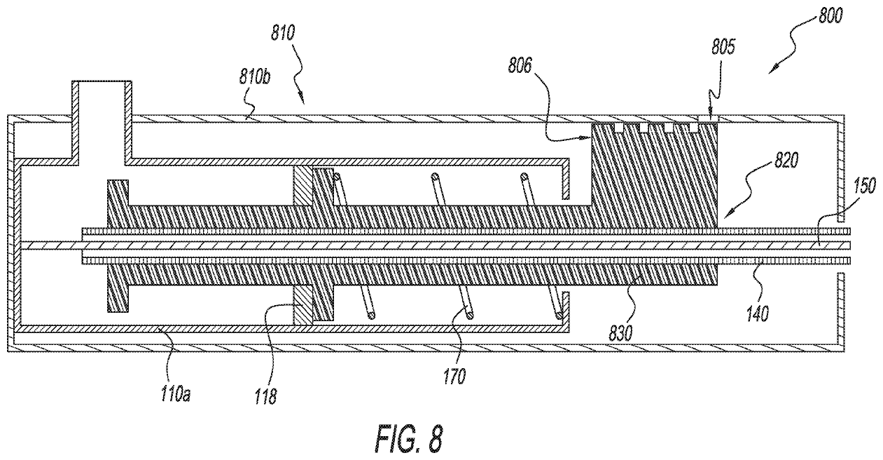

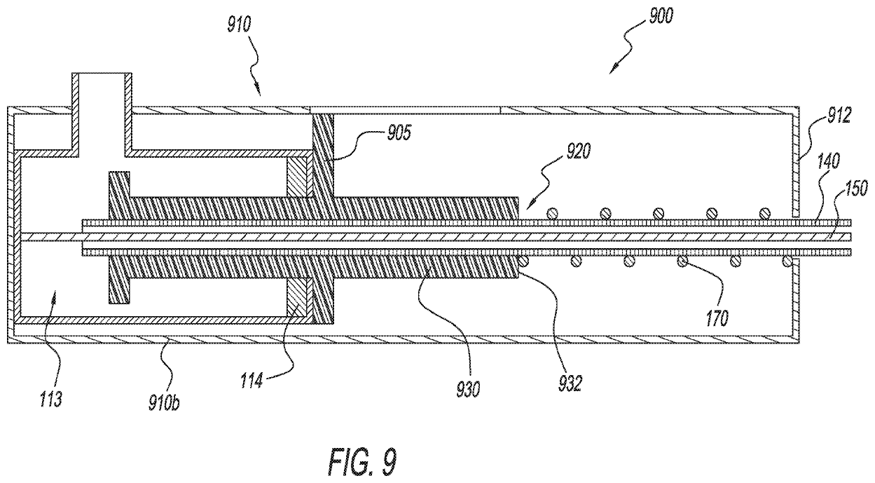

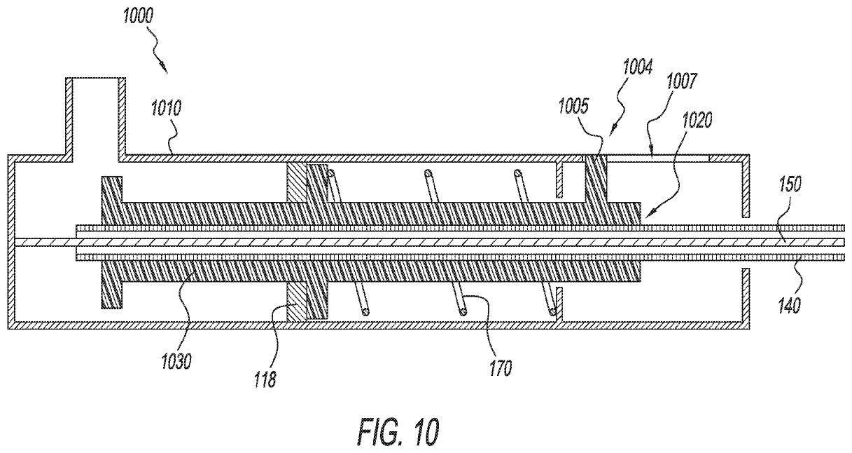

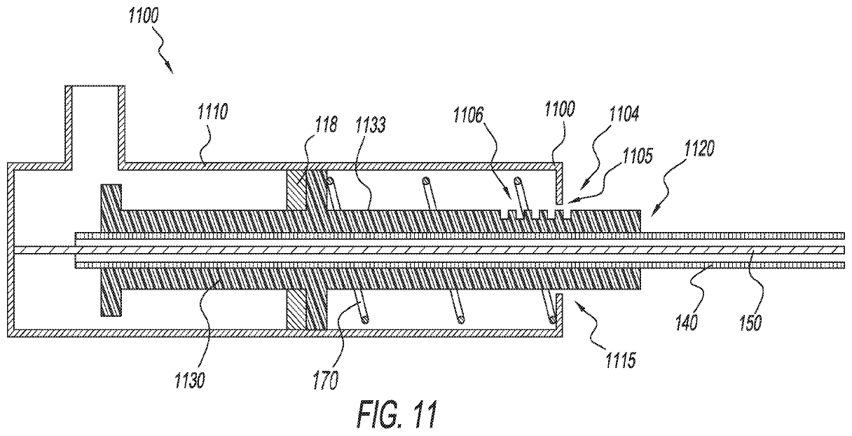

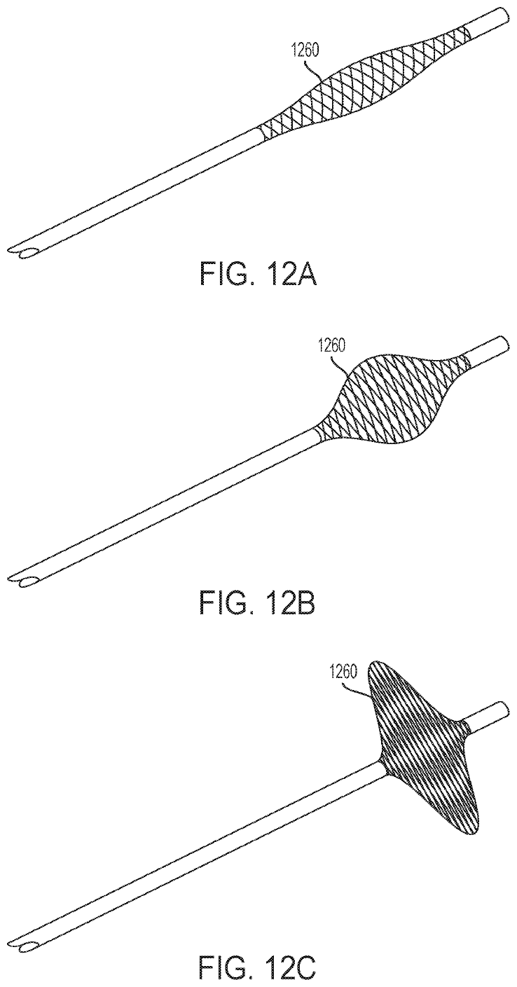

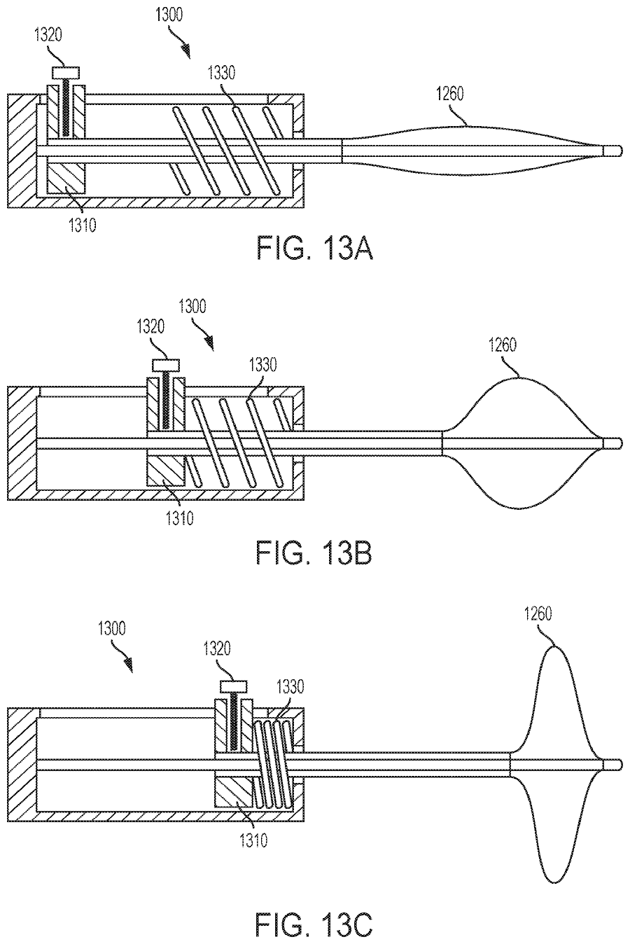

FIG. 10 shows another exemplary embodiment of an arteriotomy positioning device 1000. Device 1000 is similar to device 100, except the handle 1010 of device 1000 may differ from the handle 110 of device 100. The handle 1010 of device 1000 does not have an inner housing and an outer housing, but instead may be formed from a single housing. The indicator feature 1004 is an indication of relative movement between the catheter assembly 1020 and the handle 1010. The handle 1010 may include a window 1007 that allows the user to see the indicator feature 1005 on the catheter assembly 1020 (and preferably on the plunger 1030). The markings (not shown in FIG. 10) may be adjacent the window 1007 on an outer surface of the housing 1010.

FIG. 11 shows yet another exemplary embodiment of an arteriotomy positioning device 1100 having an alternative device status indicator 1104. Device 1100 is similar to device 1000, except the handle 1110 and catheter assembly 1120 may differ from the handle 1010 and catheter assembly 1020 of the device 1000. Specifically, the indicator feature 1105 may simply be a distal opening 1115 in the handle 1110. A series of markings 1106 on the catheter assembly 1120 (and preferably, on the outer surface 1133 of the plunger 1130) may slide relative to the distal opening 1115, and the marking 1106 that is aligned with the distal opening 1115 may indicate the status of the device 1100. The window 1007 in the handle 1010 and the protrusion that formed the indicator feature 1005 of FIG. 10 may be omitted in device 1100. This device status indicator 1104 may also be applied to other embodiments of arteriotomy positioning devices.

The device may be modified if the expandable support is not an inflatable balloon. For example, the inflation port, fluid chamber, valve, and seal in the handle may be omitted. The device may be moved from the resting state to the locating state by moving the catheter assembly distally relative to the core wire (or by moving the core wire proximally relative to the catheter assembly), which can be accomplished manually or with an actuator or control mechanism in the handle. In either case, the device should still be able to move freely between the locating state and the tension state, regardless of how the device is moved from the resting state to the locating state. The device may have one or more locking features to maintain the position of the catheter assembly. For example, a resting lock may prevent distal movement of the catheter assembly relative to the handle and the core wire when the device is in a resting state. Once the device is moved to the locating state, a locating lock may limit proximal movement of the catheter assembly relative to the handle and the core wire, while still allowing the device to move between the locating state and the tension state.

FIG. 12A is a perspective view of an alternative embodiment for an expandable support 1260 in a resting state. The expandable support 1260 is configured to be connected to an arteriotomy positioning device such as the positioning device 100 described with reference to FIGS. 1A-5C, and with a handle embodiment as described below with reference to FIGS. 13A-C.

In the embodiment depicted in FIGS. 12A-C, the expandable support 1260 is an expandable wire mesh that can move between a low-profile configuration (shown in a resting state in FIG. 12A), a medium-profile configuration (shown in a locating state in FIG. 12B), and a high-profile configuration (shown in an expanded state in FIG. 12C).

FIG. 12B is a perspective view of the arteriotomy positioning device of FIG. 12A in a locating state. As noted above, the locating state may also be referred to herein as a medium-profile configuration. If the expandable support 1260 is in the shape of a sphere or ellipsoid, its width would be the maximum diameter of the expandable support 1260 in a direction substantially perpendicularly to a longitudinal axis of the device 100. As will be described below, the length and the width of the expandable support 1260 may change as the expandable support 1260 is moved between the low-profile configuration, the medium-profile configuration, and the high-profile configuration.

The expandable support 1260 may be connected to a catheter, such as catheter 140 for example, and a core wire, such as the core wire 150 for example.

The length of the expandable support 1260 may change if a catheter assembly is moved relative to a core wire, allowing the expandable support 1260 to move between the low-profile configuration, the medium-profile configuration, and the high-profile configuration. Generally, the length may decrease and the width may increase as the expandable support 1260 moves from the low-profile configuration to the medium-profile configuration to the high-profile configuration.

FIGS. 13A-C depict an example handle 1300 comprising a plunger 1310 that may be used with the expandable support of FIGS. 12A-C. In some embodiments, the handle 1300 may comprise a number of features similar to the handle 110. Certain features specific to the embodiment of FIGS. 13A-C will be described in further detail below.

FIG. 13A depicts a cross-section view of the handle 1300 used with the expandable support of FIG. 12A in a resting state, shown from the side. In FIG. 13A, the plunger 1310 is shown in a locked position with a lock 1320 that prevents the plunger 1310 from movement, resulting in the expandable support 1260 remaining in the low-profile configuration or resting state.

In operation, once a user has inserted the device into a patient's artery, the user can unlock the lock 1320 on the plunger 1310, allowing for movement of the plunger 1310 to the position shown in FIG. 13B. FIG. 13B is a cross-section view of the handle of FIG. 13A used with the expandable support of FIG. 12B in a locating state, shown from the side.

As shown in FIG. 13B, the plunger 1310 movement in the distal direction within the handle 1300 applies force to the expandable support 1260. The applied force causes expansion of the expandable support 1260, and results in a partial expansion of the expandable support 1260, as shown in the medium-profile configuration or the locating state in FIG. 12B.

FIG. 13C is a cross-section view of the handle of FIG. 13A used with the expandable support of FIG. 12C in an expanded state, shown from the side. As the plunger 1310 moves distally within the handle 1300, the plunger contacts and engages a spring 1330 present within the handle 1300. Continual distal movement of the plunger 1310 applies pressure to and compresses the spring 1330, resulting in the configuration depicted in FIG. 13C. As the user retracts the device and the expandable support 1260 presses against the arteriotomy, tension on the plunger 1310 provides for further expansion of the expandable support 1260, resulting in the high-profile configuration shown in FIG. 12C. The spring 1320 provides friction and in turn controls the rate of expansion of the expandable support 1260.

As used herein, the relative terms “proximal” and “distal” shall be defined from the perspective of the arteriotomy positioning devices. Thus, proximal refers to the direction of the handle and distal refers to the direction of the expandable member.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein. The foregoing description is provided to enable any person skilled in the art to practice the various example implementations described herein. Various modifications to these variations will be readily apparent to those skilled in the art, and the generic principles defined herein may be applied to other implementations. All structural and functional equivalents to the elements of the various illustrious examples described throughout this disclosure that are known or later come to be known to those of ordinary skill in the art are expressly incorporated herein by reference.

What is claimed is:

1. A device for positioning an expandable support, the device comprising:
a handle;
a catheter assembly having a lumen, the catheter assembly extending from the handle;
a core wire extending from the handle through the lumen of the catheter assembly, the core wire having a proximal end connected to the handle and a distal end extending from a distal end of the catheter assembly; and
an expandable support having a proximal end connected to a distal end of the catheter assembly and a distal end connected to the distal end of the care wire;
wherein the catheter assembly is slidable relative to both the handle and the core wire.

2. The device of claim 1, wherein the expandable member is moveable between a low-profile configuration, a medium-profile configuration, and a high-profile configuration.

3. The device of claim 2, further comprising a device status indicator that indicates:
whether the expandable support is in the low-profile configuration, the medium-profile configuration, or the high-profile configuration; and
whether tension is being applied to the catheter assembly.

4. The device of claim 2, wherein a distance between the distal end of the handle and the distal end of the catheter assembly increases as the expandable member moves from the low-profile configuration to the medium-profile configuration, and the distance further increases as the expandable member moves from the medium-profile configuration to the high-profile configuration.

5. The device of claim 2, wherein a distance between the distal end of the handle and the distal end of the core wire may remain substantially constant as the expandable member moves between the low-profile configuration, the medium-profile configuration, and the high-profile configuration.

6. The device of claim 1, further comprising a spring positioned in the handle, wherein the spring applies a proximal force to the catheter assembly relative to the handle and the core wire.

7. The device of claim 1, wherein the handle comprises a fluid chamber, and an inflation port that allows communication with the fluid chamber.

8. The device of claim 7, wherein the lumen of the catheter assembly communicates with the fluid chamber.

9. The device of claim 1, wherein the catheter assembly comprises a catheter and a plunger.

10. The device of claim 1, wherein the catheter assembly comprises a resting stop that limits proximal movement of the catheter assembly relative to the handle and the core wire.

11. The device of claim 1, wherein the catheter assembly comprises a tension stop that limits proximal movement of the handle and the core wire relative to the catheter assembly.

12. A method for positioning a device adjacent to an arteriotomy of a blood vessel, the method comprising:
inserting a distal end of a device into the blood vessel, the device comprising:
a core wire connected to a handle,
a catheter assembly slidable relative to the handle and the core wire, and
an expandable support having a length, a width, a proximal end connected to the catheter assembly, and a distal end connected to the core wire, wherein the expandable support is in a low-profile configuration;
increasing the width of the expandable support and decreasing the length of the expandable support, thereby moving the expandable support from the low-profile configuration to a medium-profile configuration;
withdrawing the device proximally until the expandable support contacts a wall of the blood vessel adjacent to the arteriotomy; and
applying tension to the catheter assembly to further increase the width and decrease the length of the expandable support and bring the expandable support to a high-profile configuration.

13. The method of claim 12, wherein the relative positions of the handle and the core wire remain substantially constant during the steps of withdrawing the device proximally, and applying tension to the catheter assembly.

14. The method of claim 12, wherein the step of bringing the expandable support to the medium-profile configuration causes the catheter assembly to move distally relative to the core wire.

15. The method of claim 12, wherein the step of applying tension to the catheter assembly causes the core wire to move proximally relative to the catheter assembly.

16. The method of claim 12, wherein the device comprises a visual indicator having an indicator feature and a series of indicator markings, and wherein the step of bringing the expandable support to the medium-profile configuration causes the indicator feature to move relative to the series of indicator markings.

17. The method of claim 16, and wherein the step of applying tension to the catheter assembly causes the indicator feature to move relative to the series of indicator markings.

18. The method of claim 12, wherein the expandable support comprises a balloon, and wherein the width is a maximum diameter of the balloon.

19. The method of claim 12, wherein the expandable support comprises an expandable wire mesh.

20. The method of claim 12, wherein the step of increasing the width of the expandable support and decreasing the length of the expandable support comprises inflating the expandable support by pushing an inflation fluid through the catheter assembly and into the expandable support.

21. The method of claim 12, further comprising:
performing a procedure;
returning the device to the low-profile configuration; and
withdrawing the device from the blood vessel.

22. The method of claim 20, wherein the procedure is a vascular closure procedure.
